(19) United States
(12) Patent Application Publication
(10) Pub. No.: US 2006/0137681 A1
(43) Pub. Date: Jun. 29, 2006

(54) Actuator for a metered dose inhaler

(75) Inventors: Dirk Von Hollen, Clark, NJ (US); Eric A. Lieberman, Scotch Plains, NJ (US); Jeffrey S. N. Paine, Franklin, TN (US); Matthew T. Paine, Franklin, TN (US)

Correspondence Address: MICHAEL W. HAAS, INTELLECTUAL PROPERTY COUNSEL;RESPIRONICS, INC., 1010 MURRY RIDGE LANE, MURRYSVILLE, PA, 15668, US

(73) Assignee: RIC Investments, LLC., Wilmington, DE (US)

(21) Appl. No.: 11/315,559

(22) Filed: Dec. 22, 2005

Related U.S. Application Data
(60) Provisional application No. 60/639,852, filed on Dec. 28, 2004.

(51) Int. Cl. A61M 11/00; A61M 16/00

(52) U.S. Cl. 128/200.14; 128/200.23; 128/203.27

(57) ABSTRACT

A metered dose inhaler is provided which includes a canister fitted in an actuator body. A metered dose of medication is delivered by compressing the canister in the actuator body. The metered dose inhaler includes an actuator that either fully (automatic) or partially (user-assisted) actuates the metered dose inhaler in order to deliver medication to the user.

### CROSS-REFERENCE TO RELATED APPLICATIONS

This application claims priority under 35 U.S.C. § 119(e) from provisional U.S. patent application No. 60/639,852 filed Dec. 28, 2004 the contents of which are incorporated herein by reference.

### BACKGROUND OF THE INVENTION

1. Field of the Invention

The present invention pertains to aerosol delivery devices used to aerosolize a liquid. In particular, the present invention is related to metered dose inhalers that deliver aerosolized medication to a user.

2. Description of the Related Art

It is well known in the art that delivering medication to a user can enhance the user's quality of life. As used herein, the term medication shall be broadly interpreted to include therapeutic, prophylactic, and diagnostic agents. There are a variety of well known methods to deliver medication to a user such as orally, transdermally, or intravenously. Yet, one additional method, which is gaining in popularity, is delivering medication directly to the lungs of the user.

Delivering medication to the user's lungs may, in fact, be preferable over other traditional medication delivery methods in a variety of circumstances. For instance, this method has proven to be particularly effective in treating pulmonary diseases, such as Asthma, Chronic Obstructive Pulmonary Disease (COPD), and Cystic Fibrosis because it provides targeted administration of the medication. Delivering medication to the lungs of a user is also being considered for a variety of other conditions where the traditional delivery methods are deemed less desirable. In some cases, a user's ability to absorb medication may be compromised, or absorption may cause harm to the user's gastro-intestinal track. Delivering medication intravenously is difficult and painful for many users, and often requires the assistance of a medical professional. With respect to transdermal delivery, this method requires the user to wear an unsightly patch, and has proven to be unreliable in many instances because it depends on the patch remaining securely adhered to the user's skin in order to deliver the appropriate dose of medication.

As best appreciated by those skilled in the art, aerosolized medication may optimally target specific sites in the pulmonary system. These sites include the nasal passages, the throat, and various locations within the lung such as the bronchi, bronchioles, and alveolar regions. The ability to deliver drugs to a targeted area is largely achieved by varying the size of the medication particle, its velocity and settling properties. Particles having an aerodynamic diameter less than 2 microns are considered to be optimal for deposition in the alveolar region of the lungs. Particles that have an aerodynamic diameter of between 2 and approximately 5 microns tend to be more suitable for delivery to the bronchiole or bronchi regions. Particles with an aerodynamic size range greater than 6 microns are suitable for delivery to the laryngeal region, throat, or nasal passages.

As used herein, particles of six microns or less are referred to as “respirable” or “within the respirable range.” In turn, the percentage of the particles within a given dose of aerosolized medication that is of “respirable” size, as compared to the total dose, is referred to as the “fine particle fraction” (FPF) or “fine particle mass” (FPM) of the dose.

Many devices are used to deliver medication to the lungs, including metered dose inhalers (MDIs) and nebulizers. As seen in FIG. 1, MDI's are aerosol delivery systems with a reservoir of compressed, low boiling point liquid formulated with a medication. MDIs are designed to meter a predetermined quantity of the medication and dispense the dose as an inhalable particulate cloud.

A typical commercially available MDI is disclosed in U.S. Pat. No. 5,031,610, and is illustrated in FIG. 1. As shown in this figure, a conventional MDI 30 includes a canister 32 fitted inside a boot 34. The canister contains liquid medication in solution or suspension with a low boiling point propellant 36. The most common propellants include the chlorofluorocarbons, p-11 and p-12, and the fluorocarbons, p-134a or p-227. In addition, carbon dioxide may also be used. All of these propellants are gaseous at room temperature and standard atmospheric pressure. The canister also includes a spring, not shown, to restore the valve stem into a deactivated state after actuation by the user.

It is also known to provide the canister with a metering valve 38 for measuring a metered dose of the medication. A valve stem 40 extends from the metering valve and acts as a conduit to pass the metered dose into a nozzle block 42 in which the valve stem is seated. The nozzle block has a passageway extending through it that forms an internal chamber 44 in which the propellant formulation expands. A nozzle channel 46 is aligned with a mouthpiece portion 48 of boot 34. As the propellant expands, the medication is aerosolized and delivered into the mouthpiece portion.

To use this type of MDI, the patient places the mouthpiece portion of the boot against their lips and actuates the MDI by depressing the canister into the boot. Upon actuation, a metered dose is measured by the valve, and is expelled from the valve stem. As the patient inhales through the mouthpiece, the aerosolized medication is carried into the user's lungs. Once a metered dose of drug has been delivered, the valve stem is urged back into a deactivated state by the spring, not shown. To optimize drug delivery it has been found useful to connect a spacer tube, not shown. The spacer provides a greater fine particle dose output. In addition, it has also been found to be desirable to provide MDI's with a mask, not shown, connected to the mouthpiece or spacer

Although such devices operate effectively for their intended purpose, several advancements are still desirable. One drawback to current MDI's is that they require the user to depress the canister down into the boot in order to deliver the medication dose. The amount of force required for most MDI's has been measured to be between 5-7 pounds. Performing this operation may prove to be difficult for some users, such as the elderly or adolescents. These users may lack the strength or manual dexterity to actuate the device. In the event that the user finds it difficult to actuate the MDI, they may additionally find it difficult to synchronize their inhalation with actuation of the device. This situation is likely to result in unreliable and inconsistent medication delivery.

Accordingly, it would be desirable to have a metered dose inhaler that overcomes one or more of the disadvantages currently present in the art. It would be further desirable to have an actuator for a metered dose inhaler that requires minimal strength or dexterity to actuate. It would be still further desirable to have an actuator for a metered dose inhaler that may be used in conjunction with multiple different metered dose inhalers.

### SUMMARY OF THE INVENTION

The object of the present invention is to overcome one or more of the above noted drawbacks currently present in the art. In accordance with the broad teachings of the invention, an actuator for a metered dose inhaler is disclosed. The metered dose inhaler has a boot which holds a canister. In accordance with a first exemplary embodiment of the present invention, the actuator has a housing that holds a power source. The actuator also includes a biasable member configured to bear on the canister. The biasable member is biased by a shape memory alloy material. When power is applied across the shape memory alloy material it contracts. As the shape memory alloy material contracts, it draws the biasable member towards the canister to apply a force to the canister and deliver a dose.

In a second exemplary embodiment of the present invention, a second actuator for a metered dose inhaler is disclosed. The metered dose inhaler has a boot which holds a canister. The actuator of this embodiment has a housing that holds a power source. The actuator also includes a fixed member to provide a support and an electromagnet. The first electromagnet is magnetically coupled to a second electromagnet, or a permanent magnet, to create a magnetic force. When power is applied, a magnetic force is created to urge the canister into the boot and deliver a dose.

In a third exemplary embodiment of the present invention, a third actuator for a metered dose inhaler is disclosed. The metered dose inhaler has a boot which holds a canister. The actuator of this embodiment has a housing that holds an electrical power source. The actuator also includes a fixed member to provide a support and a piezoelectric element. When power is applied, the piezoelectric element expands to urge the canister into the boot and deliver a dose.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a cross-sectional view of a prior art metered dose inhaler;

FIG. 2 is a side perspective view of a first exemplary embodiment of a metered dose inhaler including the actuator according to the principles of the present invention;

FIG. 3 is an exploded perspective view of the MDI of FIG. 1 depicting the canister and boot.

FIG. 4 is an exploded perspective view of the MDI of FIG. 1 depicting the boot, canister, and actuator;

FIG. 5 is a schematic circuit diagram of the actuator in the MDI of FIG. 1;

FIG. 6 is a top perspective view of the MDI of FIG. 1;

FIG. 7 is a partial perspective view of the MDI of FIG. 1;

FIG. 8A is a side elevational view of a second exemplary embodiment of an MDI according to the principles of the present invention showing the canister in a deactivated state;

FIG. 8B is a side elevational view of the MDI of FIG. 8A showing the canister in an activated state;

FIG. 9A is a schematic circuit diagram of the second exemplary embodiment of the actuator in the MDI of FIGS. 8A and 8B with two electromagnets;

FIG. 9 B is a schematic circuit diagram of the second exemplary embodiment of the actuator in the MDI of FIGS. 8A and 8B with one electromagnet and one permanent magnet.

FIG. 10 is a side elevational view of a third exemplary embodiment of an MDI according to the principles of the present invention; and

FIG. 11 is a schematic circuit diagram of the third exemplary embodiment of the actuation in the MDI of FIG. 10.

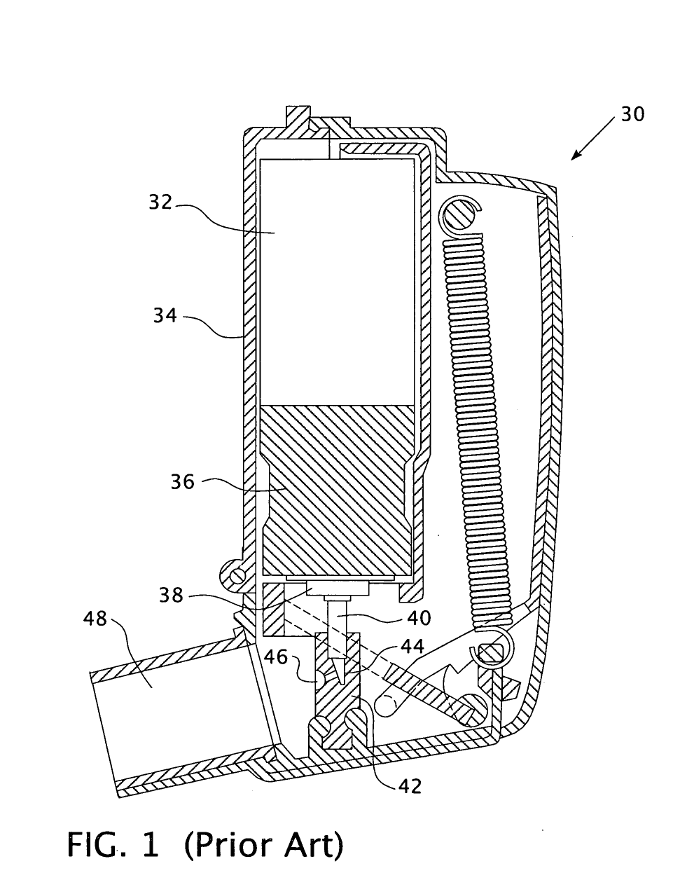

### DETAILED DESCRIPTION OF THE PRESENTLY PREFERRED EMBODIMENTS OF THE INVENTION

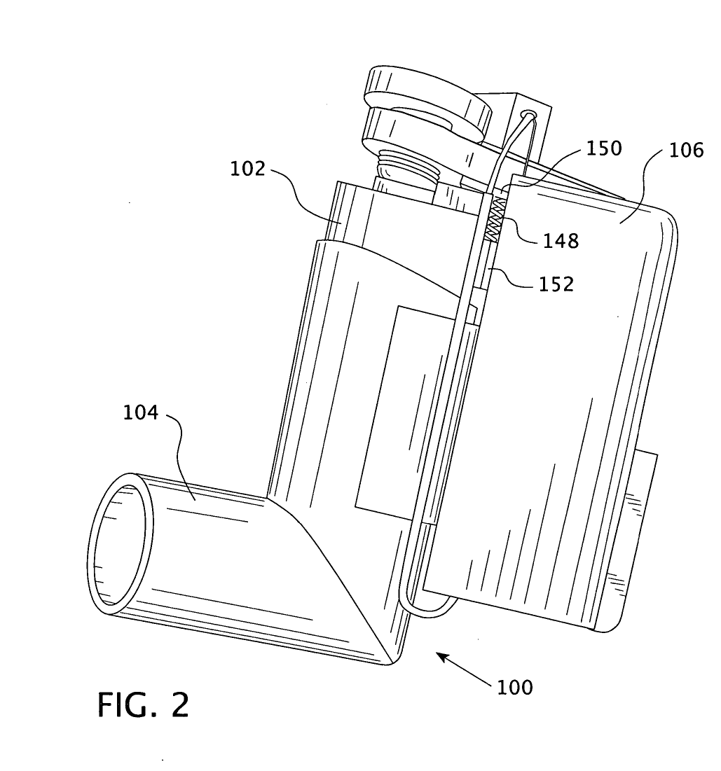

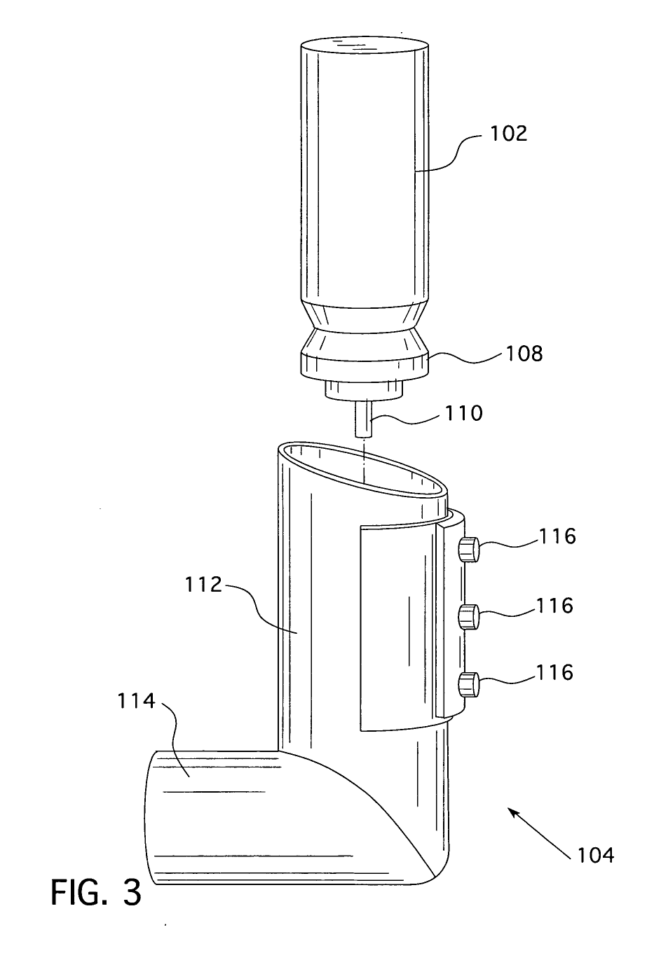

FIG. 2 illustrates an exemplary embodiment of a metered dose inhaler 100 according to the principles of the present invention. The metered dose inhaler includes a canister 102 fitted into a boot 104 and an actuator 106. The canister 102 holds a liquid having medication and propellant. As seen in FIG. 3, the canister has a generally cylindrical shape. Canister 102 terminates at a metering valve 108 connected to a biasable valve stem 110. As is well known in the art, when the valve stem is depressed, a metered dose of aerosolized medication is expelled from the canister and out the valve stem 110.

The boot is fitted about a portion of the canister and includes a canister receptacle portion 112 and a mouthpiece portion 114. The canister receptacle portion receives at least a portion of the canister, and the mouthpiece portion is configured to be inserted into a user's mouth. Canister receptacle portion 112 is sized slightly larger than the outside diameter of canister 102 so that canister 102 can be linearly slid relative to boot 104. As the canister is depressed down into the canister receptacle portion of the boot, valve stem 110 is actuated to dispense a dose of medication. As the medication and propellant are exposed to the external environment, the propellant expands into a gas with the medication suspended in the gas creating a medication cloud. As the user inhales, the medication cloud is drawn into the user's respiratory system.

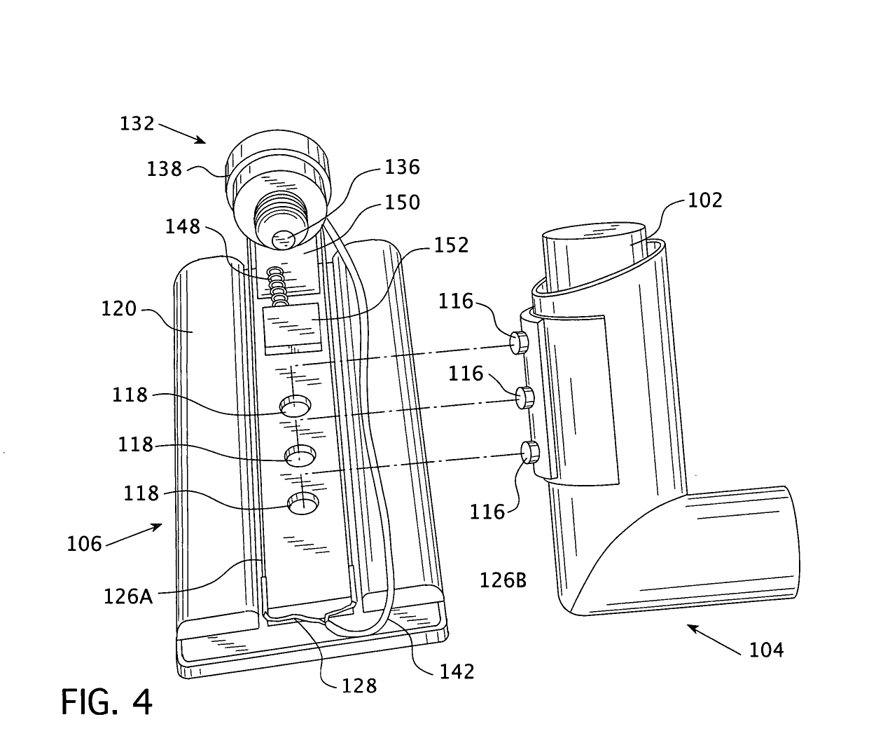

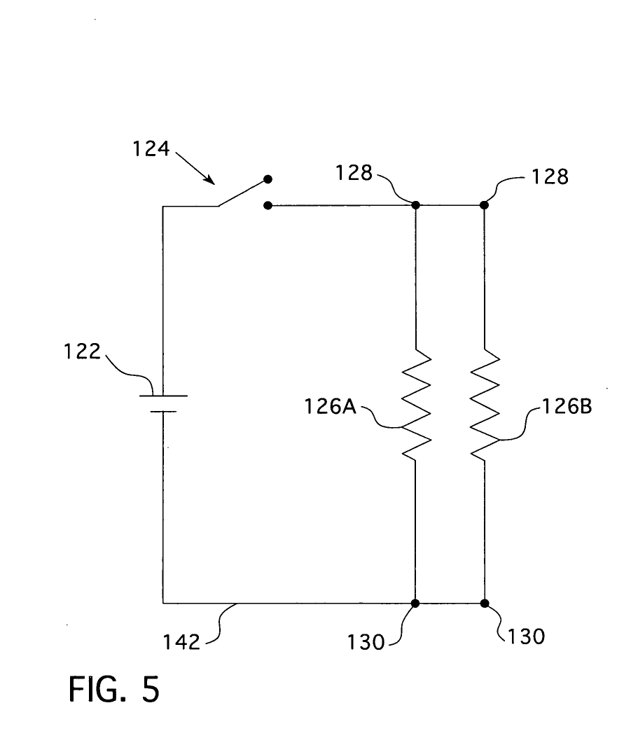

As seen in FIGS. 4 and 5, the metered dose inhaler includes an actuator 106 that is coupled to the boot. Boot 104 has bosses 116 which are fitted into holes 118. Of course a variety of connection methods could be used to interconnect actuator 106 and boot 104. Alternatively, the actuator may be formed together integrally with the boot to simplify the manufacturing process.

The purpose of the actuator is to apply a compressive force to the canister to deliver a dose of medication. This reduces or replaces the force that is manually applied by the user in contemporary devices. The present invention contemplates that the actuator may be configured to fully actuate the canister by applying the full force needed or that for some applications it may be desirable to have a patient-assisted actuator, which delivers a portion of the total required force, thus allowing the patient to supply the remaining force needed to actuate the device. The patient-assisted actuator may be desirable to conserve battery life or reduce the size of the components. It may also have the added benefit of allowing the user to maintain some manual control and prevent inadvertent actuation.

It has been found that a force of between 5-7 pounds is needed to actuate most contemporary canisters. As noted above, the actuator of the present invention may be configured to supply the entire 5-7 pounds needed, or it could be configured to supply some portion less than the total required, such as 3-4 pounds. In order to activate the device in this second example, the user would need to supply the remaining 1-4 pounds to actuate the canister.

The actuator has a housing 120 that holds a power source, such as a battery 122. However, the power source may be any power source capable of delivering electrical power including alternating current (AC) or direct current (DC) sources. The battery, shown in FIG. 5, may be any contemporary DC battery capable of providing the required power, and may be single use or rechargeable. A switch 124 and a shape memory alloy material 126A, 126B is operatively connected in parallel relative to each other. It should be noted that FIG. 5 shows two shape memory alloy materials (each of which is connected in series with the switch and the power source). It can be appreciated that the present invention also contemplates using only one shape memory alloy material or using more than two, as desired. In addition, if more than one shape memory alloy material is used, they can be connected in series with one another, in parallel with one another, or both.

The shape memory alloy material is a type of material that can change its mechanical characteristics in response to being exposed to an elevated temperature. If heated above its transition temperature the material goes through a phase shift from martinsite to austinsite. The material may be heat treated to “store” a particular shape. Above a given transition temperature, the material has a memory and will attempt to regain the stored shape. Below its transition temperature, the material will become ductile. Some shape memory alloy materials may have two transition temperatures so that two material shapes can be stored. These shape memory alloy materials may be heated directly, or may be heated by passing a current through the material and relying on resistive heating.

There are a variety of shape memory alloys currently available and suitable for use in the present invention, such as Nickel-Titanium, Copper-Aluminum-Nickel, Copper-Zinc-Aluminum, and Iron-Manganese-Silicon. In an exemplary present embodiment, a Nickel-Titanium alloy, sold by Dynalloy, Inc. under the trademark Flexinol™, has been utilized. This material has a diameter of 0.005″ with a transition temperature of 90 degrees Celsius. As used in this application, the Flexinol™ material utilized has a length of approximately 4 inches. Of course, a variety of other lengths may be used.

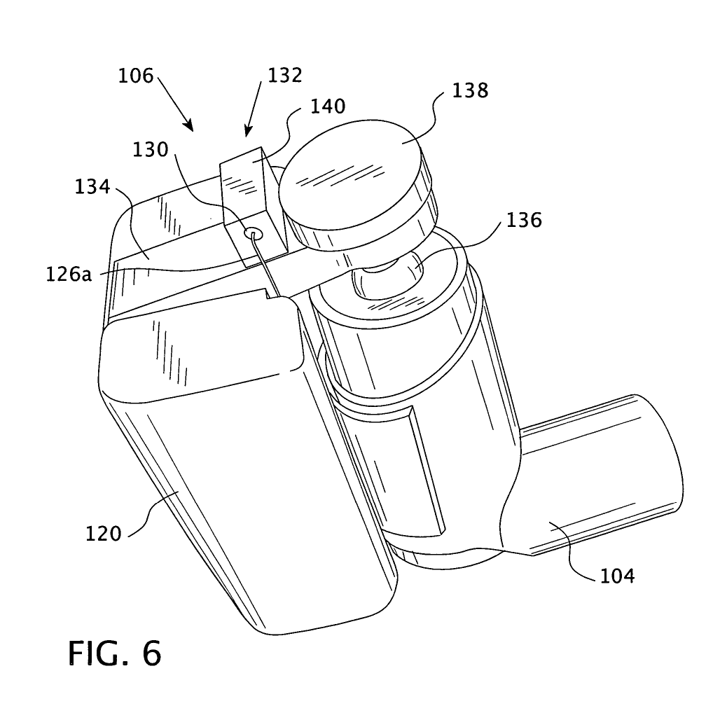

The actuator 106 includes a biasable device 132 that is capable of moving relative to housing 120 to apply a force on canister 102. One of ordinary skill in the art can best appreciate that several different biasable devices could be utilized without departing from the scope of the present invention. As seen in FIG. 6, the biasable device is a cantilevered arm 134 pivotably attached to housing 120. Cantilevered arm 134 includes a contact portion 136 and a button 138. The present invention also contemplates that the biasable device 132 may be slidably connected to the housing 120. This configuration permits the biasable device to move in a telescoping relationship relative to the housing 120. Of course, any configuration which allows for relative motion between the biasing device and the canister may be utilized. In addition, housing 120 and boot 104 could be formed together integrally.

Button 138 extends from cantilevered arm 134 and is configured to permit the user to apply a manual force on the cantilevered arm 134. The button permits the user to apply an additional force in the event that cantilevered arm 134 applies only a portion of the force needed to actuate canister 102. In addition, this feature of the invention provides a secondary backup in case actuator 106 is inoperable. This may be particularly beneficial in the event that the battery is not capable of providing an adequate electrical power or if there is a mechanical failure. The button may be operated until the actuator is fixed or replaced.

The shape memory alloy material is secured between housing 120 and biasable member 132. The shape memory alloy material is secured at one end to housing 120 by terminals 128. The other end of the shape memory alloy material is attached to the biasable member 132 with terminals 130. Terminals 130 are held in place by support 140. The support retains terminals 130 and houses an electrical connector 142. Terminals 128 are spaced apart approximately 2.3 inches from terminals 130. In order to increase the amount of force that can be exerted by the shape memory alloy material, the shape memory alloy material 126A, 126B is pair of wires made from a shape memory alloy material to double the amount of force generated. One skilled in the art can best appreciate that the amount of force exerted can be modified by utilizing more or less shape memory alloy material, by changing the material composition, or by changing the physical dimensions of the shape memory alloy material utilized. Having the terminals 128 and 130 spaced apart by 2.3 inches has been found to provide the optimal force for this application. However, one skilled in the art can best appreciate that the amount of force exerted can be adjusted by varying the length between terminals 128, 130.

The switch 124, schematically represented in FIG. 5, may be any of a multitude of switches commonly known in the art. In one embodiment of the present invention, the switch is any manual switch, such as a push button switch, slid switch, or an elastomeric membrane switch. These switches are manually operated by the user. In another exemplary embodiment of the present invention, switch 124 is an automatic switch that is activated upon sensing that the user has begun inhaling. In this embodiment, switch 124 is a pressure activated switch utilizing a diaphragm, piston, or piezoelectric sensor located in or near the mouthpiece to detect airflow. Alternatively, airflow switches could also be used. Airflow switches commonly use a vane or temperature sensor to detect airflow such as a hot-wire or a thermistor based anemometer. As the vane moves or the temperature changes due to airflow, switch 124 would be activated. Of course even if an automatic switch is utilized a mechanical switch may also be included to act as a backup in the event that the automatic switch has malfunctioned. Whether a mechanical switch or automatic switch is utilized, the switch operatively connects the power source across the shape memory alloy material 126A, 126B.

Actuator 106 may also include a return mechanism 144 having a biasing device 146 to supplement the restorative force exerted by the spring, not shown, in the canister 102. The return mechanism will assist in urging the biasing member back away from the canister once actuation has completed. As seen in FIG. 7, the return mechanism is a spring 148 fitted between first bearing surface 150 and second bearing surface 152. First bearing surface 150 extends from cantilevered arm 134, and second bearing surface 152 extends from the housing. In addition, the device can be designed to utilize a shape memory alloy material with two transition temperatures: one at or near room temperature and one at a temperature above 50 degrees Celsius. Rather than using a return mechanism, two shapes could be “stored” in the material's shape memory. The first one will have a shape which would tend to curl and draw the cantilevered arm down on the canister. The other would store a shape that would straighten and urge the cantilevered arm away from the canister.

In order to dispense a dose, the user will actuate switch 124 (either manually or automatically). The switch will permit current to flow through shape memory alloy 126A, 126B. As the current flows through the shape memory alloy, its temperature will rise until it passes through the threshold phase temperature of the material. Once the temperature of the shape memory alloy is above its transition temperature, the shape memory alloy 126A, 126B will contract. Because the shape memory alloy is securely held by terminals 128, 130, the contraction of this material will result in the cantilevered arm being drawn downward and apply a force on canister 102 to dispense a dose.

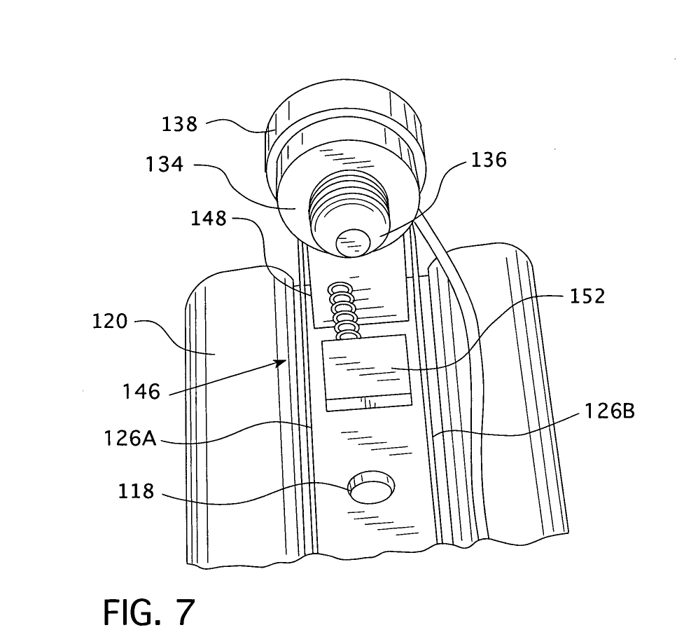

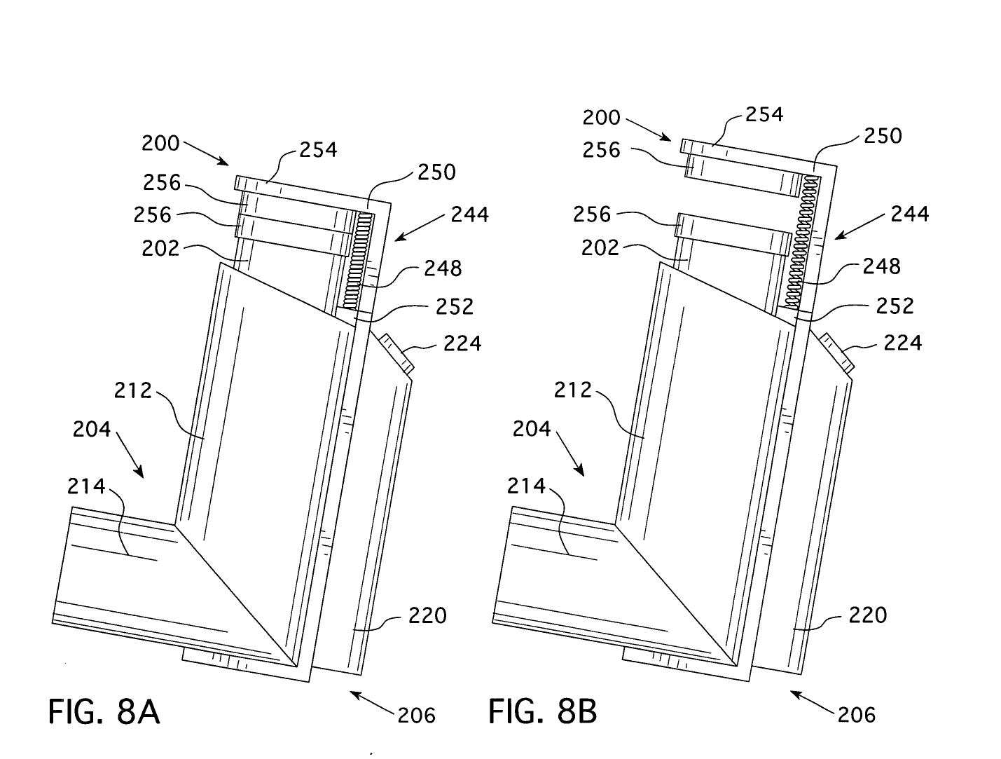

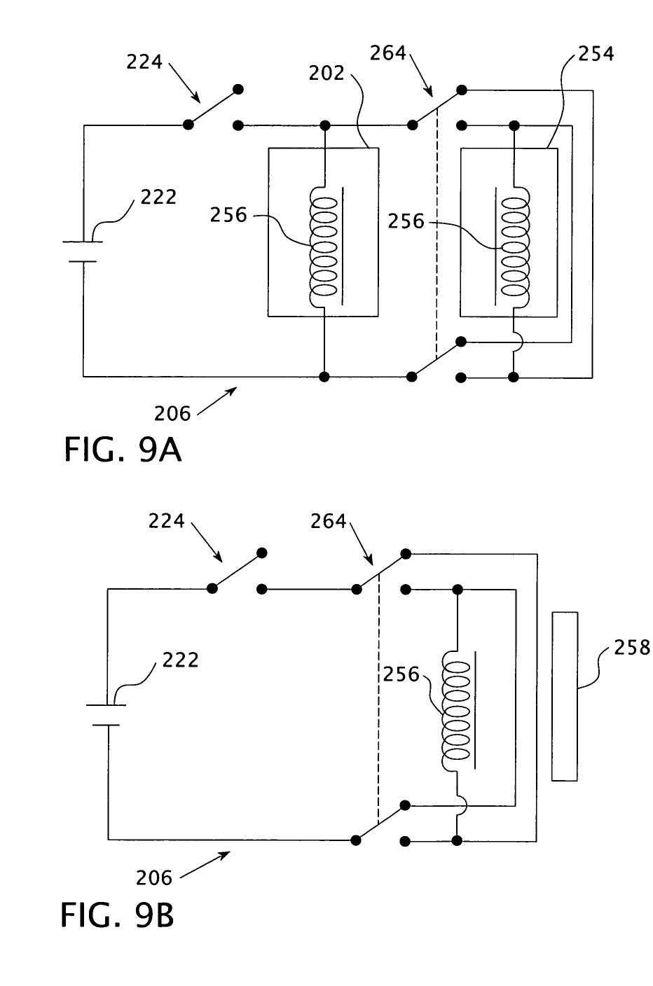

In another exemplary embodiment, the actuator relies on an electromagnet to create the force necessary to actuate the canister. In this embodiment, as seen in FIGS. 8A-9B, MDI 200 once again has a canister 202, a boot 204, and an actuator 206. The boot 204 has a canister receptacle portion 212 and a mouthpiece portion 214. Actuator 206 includes a housing 220 which provides a receptacle for battery 222 (shown in FIG. 9A, 9B) and a switch 224. Switch 224 is shown in a deactivated state in FIG. 8A and an activated state in FIG. 8B. Switch 224 may be automatic or manual as described above with respect to switch 124 in the previous embodiment. As best appreciated with reference to FIGS. 9A and 9B, actuator 206 includes a battery 222 connected in series with switch 224 and at least one electromagnet 256. Once again, the switch may be manually or automatically actuatable as described above with reference to the first exemplarily embodiment.

The present invention may utilize two electromagnets 256, as seen in FIG. 9A. Rather than utilizing a biasable member, this embodiment has a fixed member 254. One electromagnet 256 is affixed to the fixed member 254; another electromagnet 256 is affixed to canister 202. The electromagnets are oriented in close proximity to one another such that application of power source will create an electromagnetic field. The electromagnets are wound such that the electrical potential results in a repulsive force that will urge the magnets away from one another to actuate the MDI. Of course, the actuator could be reconfigured so that an attractive force between the magnets would result in actuation of the MDI.

Because one electromagnet is affixed to the canister and the other electromagnet is affixed to the fixed member, the repulsive force created will result in urging canister 202 away from the fixed member thereby actuating the MDI. Alternatively, as seen in FIG. 9B, this invention may also utilize a single electromagnet attached to either the fixed member 254 or the canister 202. To create a force between the canister 202 and the fixed member 254, the other element (either the canister or the fixed member) will have a permanent magnet 258. The electromagnet and the permanent magnet create a repulsive electromotive force between the canister and the fixed member to actuate the MDI.

This exemplary embodiment may also include a return mechanism 244. The return mechanism may include a biasing device 246, such as spring 248. The spring is fitted between a first surface 250 located on the fixed member 254 and a second surface 252 located on housing 220. Alternatively, the return mechanism may be a selector switch 264 shown in FIGS. 9A and 9B. The selector switch allows for the polarity of the electrical potential across one of the electromagnets to be reversed, thus reversing the polarity of the electromagnetic field produced by the electromagnet. This will result in an attractive force, rather than a repulsive force, to draw the canister back from the boot.

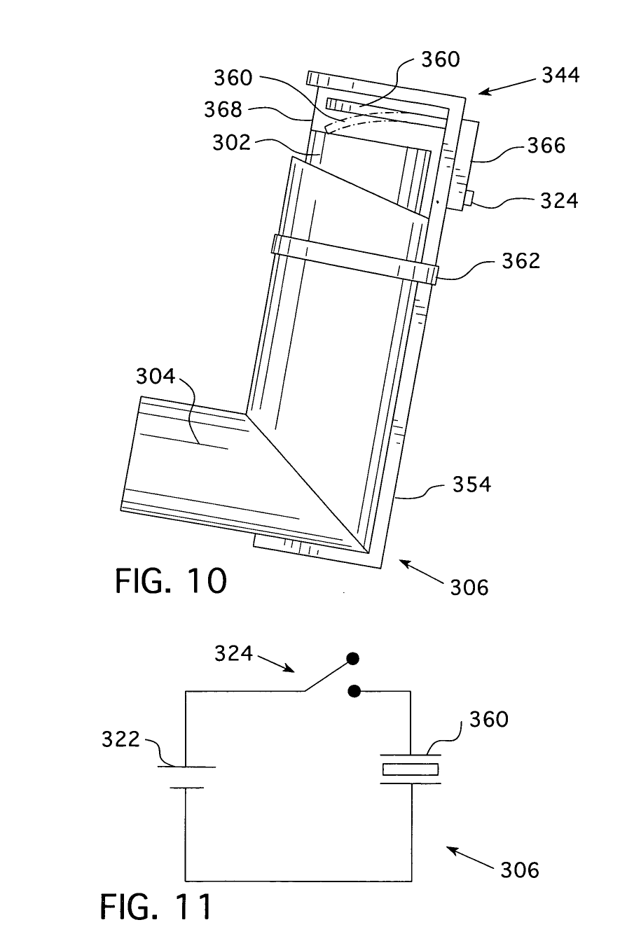

In yet another exemplary embodiment of the present invention, as best appreciated with reference to FIG. 10, MDI 300 includes a canister 302, a boot 304, and an actuator 306. The actuator of this embodiment uses a piezoelectric element 360 disposed on a fixed member 354. When current is supplied to the piezoelectric element, the piezoelectric element curls downward and applies a force on canister 302. The actuator is secured to boot 304 by a hook and loop strap 362 wrapped around boot 304 and actuator 306.

As is well known, piezoelectric elements create an electrical current when pressure is exerted on the element. These piezoelectric elements are often used as pressure sensors. It is also well known that piezoelectric elements will curl when current is supplied to the element. It is this characteristic of piezoelectric elements that is utilized in the present invention. As seen in FIG. 11, the actuator 306 has a battery 322 connected in series with a switch 324 and a piezoelectric element 360. Once again, switch 324 may be manual or automatic as described above with respect to switches 124 and 224 in the previous embodiments. When current is applied to the piezoelectric element, the shape of the piezoelectric element will change.

The MDI has a display 366. The display may be used to relay information to the user, such as activation status, doses delivered, etc. This embodiment also contemplates the use of a skirt 368 that fits about piezoelectric element 360 to isolate the piezoelectric element from the external environment. The piezoelectric element may be urged back into its deactivated state by merely disconnecting the battery from the piezoelectric element. Further, the MDI may also include a return mechanism 344 having a biasing device 346 such as spring 348.

In use the user of the present invention is provided with an enhanced method of actuatating a canister. This device provides several advantages over contemporary metered dose inhalers. The present invention utilizes electrical components to create a force to actuate the canister. This feature is particularly advantageous for user's who may lack the strength or manual dexterity to actuate the device manually. However, as a safety feature, the canister may still be actuated manually in the event that the actuator has malfunctioned.

Although the invention has been described in detail for the purposes of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. In addition, specific features of this invention are shown in some drawings and not others, this is for convenience only as each feature may be combined with any or all of the other features in accordance with the invention. Other embodiments will occur to those skilled in the art and are within the following claims:

What is claimed is:

1. An actuator for a metered dose inhaler, the actuator comprising:
a housing;
a biasable member;
a shape memory alloy material connected to the biasable member;
a power source; and
a switch, wherein the switch has at least two positions, a first position wherein the power source is operatively connected to the shape memory alloy material, and a second position wherein the power source is disconnected from the shape memory alloy material, and wherein the shape memory alloy material biases the biasing member towards the canister responsive to the switch being in the first position.

2. A metered dose inhaler for use in delivering an aerosol to a user, the metered dose inhaler comprising:
a canister having a metering valve and a valve stem extending from the metering valve;
a boot having a canister receptacle portion and a mouthpiece receptacle portion; and
an actuator located proximate the canister and boot, the actuator including a housing, a biasable member, a shape memory alloy material connected to the biasable member, a power source and a switch, wherein the switch has at least two positions, a first position wherein the power source is connected to the shape memory alloy material and a second position wherein the power source is disconnected from the shape memory alloy material, wherein the shape memory alloy material biases biasing member towards the canister when the switch is in the first position.

3. The metered dose inhaler as recited in claim 2, wherein the shape memory alloy material is at least one wire connected between the biasable member and the housing.

4. The metered dose inhaler as recited in claim 3, wherein the biasable member is a cantilevered arm extending from the housing.

5. The metered dose inhaler as recited in claim 4, wherein the actuator further comprises a return mechanism

6. The metered dose inhaler as recited in claim 5, wherein the return mechanism further comprises:
a first surface on the cantilevered arm;
a second surface on the housing; and
a biasing device located between the first surface and the second surface.

7. An actuator for a metered dose inhaler, the actuator comprising:
a housing;
a fixed member connected to the housing;
a first electromagnet;
a power source; and
a switch having a first position wherein the power source is connected to the first electromagnet and a second position wherein the power source is disconnected from the first electromagnet.

8. The actuator as recited in claim 7, wherein the actuator further comprises a second electromagnet, and wherein the power source is connected to the second electromagnet in the first position and is disconnected from the second electromagnet in the second position.

9. A metered dose inhaler for use in delivering an aerosol to a user, the metered dose inhaler comprising:
a canister having a metering valve and a valve stem extending from the metering valve;
a boot having a canister receptacle portion and a mouthpiece receptacle portion; and
an actuator for a metered dose inhaler, the actuator including a housing, a fixed member, at least one electromagnet, a power source, and a switch having a first position wherein the power source is connected to the at least one electromagnet and a second position wherein the power source is disconnected from the at least one electromagnet.

10. The metered dose inhaler as recited in claim 9, wherein the metered dose inhaler further comprises a permanent magnet located proximate to the electromagnet.

11. The metered dose inhaler as recited in claim 9, wherein the actuator further comprises a return mechanism.

12. The metered dose inhaler as recited in claim 1 1, wherein the return mechanism further comprises:
a first surface on the fixed member;
a second surface on the housing; and
a biasing device located between the first surface and the second surface.

13. The metered dose inhaler as recited in claim 12, wherein the biasing device is a spring.

14. The metered dose inhaler as recited in claim 12, wherein the return mechanism further comprises a switch, wherein the switch is connected to reverse the flow direction of the power source in at least one of the electromagnets.

15. An actuator for a metered dose inhaler, the actuator comprising:
a housing;
a fixed member;
at least one piezoelectric element;
a power source; and
a switch having a first position wherein the power source is connected to the at least one piezoelectric element and a second position wherein the power source is disconnected from the at least one piezoelectric element.

16. A metered dose inhaler for use in delivering an aerosol to a user, the metered dose inhaler comprising:
a canister having a metering valve and a valve stem extending from the metering valve;
a boot having a canister receptacle portion and a mouthpiece receptacle portion; and
an actuator for a metered dose inhaler, the actuator including a housing, a fixed member, at least one piezoelectric element, a power source, and a switch having a first position wherein the power source is connected to the at least one piezoelectric element and a second position wherein the power source is disconnected from the at least one piezoelectric element.

17. The metered dose inhaler as recited in claim 16, wherein the actuator further comprises a return mechanism.
